(19) United States
(12) Patent Application Publication
(10) Pub. No.: US 2023/0095710 A1
(43) Pub. Date: Mar. 30, 2023

(54) ENRICHMENT ENHANCERS FOR TASTE IMPROVEMENT

(71) Applicant: Blue California, Rancho Santa Margarita, CA (US)

(72) Inventors: Kathy OGLESBY, Rancho Santa Margarita, CA (US); Aaron Jun KAMIMURA, Irvine, CA (US)

(73) Assignee: Blue California, Rancho Santa Margarita, CA (US)

(21) Appl. No.: 17/855,354

(22) Filed: Jun. 30, 2022

Related U.S. Application Data
(60) Provisional application No. 63/217,702, filed on Jul. 1, 2021.

(51) Int. Cl. A23L 27/20; C07D 313/00; A23L 27/00; A23J 3/22; A23C 11/00

(52) U.S. Cl. CPC A23L 27/2052; C07D 313/00; A23L 27/88; A23J 3/227; A23C 11/00; A23V 2002/00

(57) ABSTRACT

The present disclosure provides compositions comprising delta lactones for use in improving taste in food products.

### BACKGROUND OF THE INVENTION

### Field of Invention

The present disclosure provides compositions comprising delta lactones for use in improving taste in food products.

### Background

Lactones are used in the food industry as additives to provide additional texture, flavor, and richness to food products. However, blending two or more lactones in a single composition may result in unpredictable and/or undesirable tastes. There exists a need for optimal blends of two or more lactones that provide favorable taste profiles.

### BRIEF SUMMARY OF THE INVENTION

In one aspect, the disclosure provides compositions comprising:

(a) a compound of Formula I:

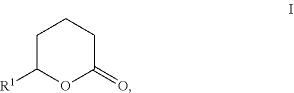

or a stereoisomer thereof, wherein R¹ is C₈-C₁₆ alkyl; and

(b) a compound of Formula II:

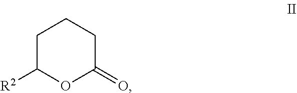

or a stereoisomer thereof, wherein R² is C₈-C₁₆ alkyl,

with the proviso that R¹ does not equal R².

In another aspect, the disclosure provides compositions comprising:

(a) a compound of Formula I:

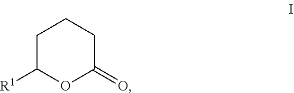

or a stereoisomer thereof, wherein R¹ is C₈-C₁₆ alkyl;

(b) a compound of Formula II:

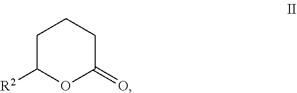

or a stereoisomer thereof, wherein R² is C₈-C₁₆ alkyl,

with the proviso that R¹ does not equal R²; and

(c) one or more compounds of Formula III:

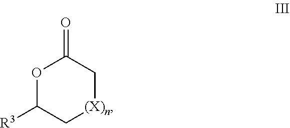

or geometric isomers and/or stereoisomers thereof, wherein:

each X is independently —CH₂—, —CH═CH—CH₂—, or —C≡C—CH₂—;

R³ is C₁-C₆ alkyl; and

n is an integer between 6-30, inclusive.

In another aspect, the disclosure provides methods of enhancing the flavor, texture, or richness of a plant-based meat product or plant-based dairy product comprising admixing a composition comprising:

(a) a compound of Formula I:

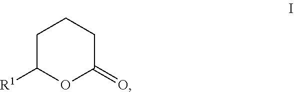

or a stereoisomer thereof, wherein R¹ is C₈-C₁₆ alkyl; and

(b) a compound of Formula II:

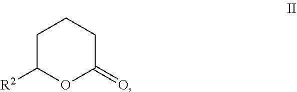

or a stereoisomer thereof, wherein R² is C₈-C₁₆ alkyl,

with the proviso that R¹ does not equal R²;

with the plant-based meat or plant-based dairy product.

In another aspect, the disclosure provides methods of enhancing the flavor, texture, or richness of a plant-based meat product or plant-based dairy product comprising admixing a composition comprising:

(a) a compound of Formula I:

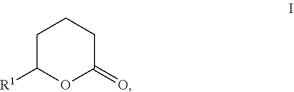

or a stereoisomer thereof, wherein R¹ is C₈-C₁₆ alkyl; and

(b) a compound of Formula II:

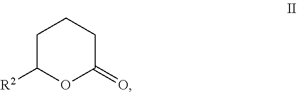

or a stereoisomer thereof, wherein R² is C₈-C₁₆ alkyl,

with the proviso that R¹ does not equal R²; and

(c) one or more compounds of Formula III:

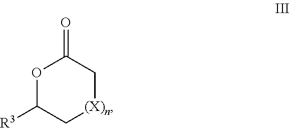

or geometric isomers and/or stereoisomers thereof, wherein:

each X is independently —CH₂—, —CH═CH—CH₂, or —C≡C—CH₂—;

R³ is C₁-C₆ alkyl; and

n is an integer between 6-30, inclusive.

In another aspect, the disclosure provides compositions comprising:

(a) a compound of Formula I:

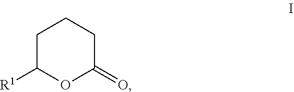

or a stereoisomer thereof, wherein R¹ is C₈-C₁₆ alkyl; and

(b) a compound of Formula II:

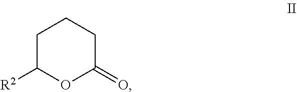

or a stereoisomer thereof, wherein R² is C₈-C₁₆ alkyl,

with the proviso that R¹ does not equal R²;

for use in enhancing the flavor, texture, or richness of a plant-based meat product or plant-based dairy product.

In another aspect, the disclosure provides compositions comprising:

(a) a compound of Formula I:

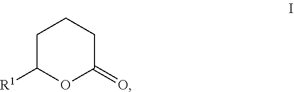

or a stereoisomer thereof, wherein R¹ is C₈-C₁₆ alkyl; and

(b) a compound of Formula II:

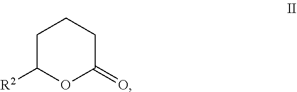

or a stereoisomer thereof, wherein R² is C₈-C₁₆ alkyl,

with the proviso that R¹ does not equal R²; and

(c) one or more compounds of Formula III:

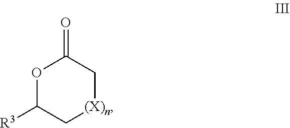

or geometric isomers and/or stereoisomers thereof, wherein:

each X is independently —CH₂—, —CH═CH—CH₂, or —C≡C—CH₂—;

R³ is C₁-C₆ alkyl; and

n is an integer between 6-30, inclusive,

for use in enhancing the flavor, texture, or richness of a plant-based meat product or plant-based dairy product.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### DETAILED DESCRIPTION OF THE INVENTION

### Compositions

In one embodiment, the disclosure provides compositions comprising:

(a) a compound of Formula I:

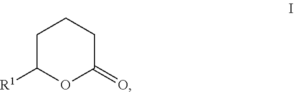

or a stereoisomer thereof, wherein R¹ is C₈-C₁₆ alkyl; and

(b) a compound of Formula II:

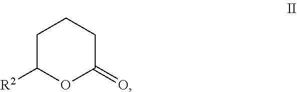

or a stereoisomer thereof, wherein R² is C₈-C₁₆ alkyl,

with the proviso that R¹ does not equal R².

In another embodiment, the composition comprises a compound of Formula I and a compound of Formula II, wherein R¹ and R² are any of the combinations set forth in Table 1:

TABLE 1

| R¹ | R² |
|---|---|
| C₈ alkyl | C₉ alkyl |
| C₈ alkyl | C₁₀ alkyl |
| C₈ alkyl | C₁₁ alkyl |
| C₈ alkyl | C₁₂ alkyl |
| C₈ alkyl | C₁₃ alkyl |
| C₈ alkyl | C₁₄ alkyl |
| C₈ alkyl | C₁₅ alkyl |
| C₈ alkyl | C₁₆ alkyl |
| C₉ alkyl | C₁₀ alkyl |
| C₉ alkyl | C₁₁ alkyl |
| C₉ alkyl | C₁₂ alkyl |
| C₉ alkyl | C₁₃ alkyl |
| C₉ alkyl | C₁₄ alkyl |
| C₉ alkyl | C₁₅ alkyl |
| C₉ alkyl | C₁₆ alkyl |
| C₁₀ alkyl | C₁₁ alkyl |
| C₁₀ alkyl | C₁₂ alkyl |
| C₁₀ alkyl | C₁₃ alkyl |
| C₁₀ alkyl | C₁₄ alkyl |
| C₁₀ alkyl | C₁₅ alkyl |
| C₁₀ alkyl | C₁₆ alkyl |
| C₁₀ alkyl | C₁₂ alkyl |
| C₁₀ alkyl | C₁₃ alkyl |
| C₁₁ alkyl | C₁₄ alkyl |
| C₁₁ alkyl | C₁₅ alkyl |
| C₁₁ alkyl | C₁₆ alkyl |
| C₁₁ alkyl | C₁₃ alkyl |
| C₁₂ alkyl | C₁₄ alkyl |
| C₁₂ alkyl | C₁₅ alkyl |
| C₁₂ alkyl | C₁₆ alkyl |
| C₁₃ alkyl | C₁₄ alkyl |
| C₁₃ alkyl | C₁₅ alkyl |
| C₁₃ alkyl | C₁₆ alkyl |
| C₁₄ alkyl | C₁₅ alkyl |
| C₁₄ alkyl | C₁₆ alkyl |
| C₁₅ alkyl | C₁₆ alkyl |

In another embodiment, R¹ is C₉ alkyl.

In another embodiment, the compound of Formula I is:

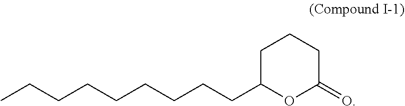 [SMILES: CCCCCCCCCC1CCCC(=O)O1]

In another embodiment, R² is C₁₁ alkyl.

In another embodiment, the compound of Formula II is:

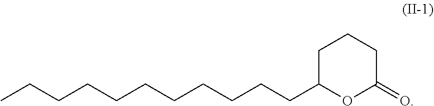 [SMILES: CCCCCCCCCCCC1CCCC(=O)O1]

In another embodiment, the compound of Formula I is racemic, i.e., optically inactive.

In another embodiment, the asymmetric carbon atom of Formula I is in the S configuration.

In another embodiment, the asymmetric carbon atom of Formula I is in the R configuration.

In another embodiment, the compound of Formula II is racemic, i.e., optically inactive.

In another embodiment, the asymmetric carbon atom of Formula II is in the S configuration.

In another embodiment, the asymmetric carbon atom of Formula II is in the R configuration.

In another embodiment, the ratio of the compound of Formula I to the compound of Formula II in the composition is about 1:1 to about 12:1, e.g., about 1:1, about 1.5:1, about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, about 10:1, about 10.5:1, about 11:1, about 11.5:1, or about 12:1.

In another embodiment, the ratio of the compound of Formula I to the compound of Formula II is about 2.8:1.

In another embodiment, the ratio of the compound of Formula I to the compound of Formula II is about 1.25:1.

In another embodiment, the ratio of the compound of Formula I to the compound of Formula II is about 6.7:1.

In another embodiment, the composition comprising a compound of Formula I and a compound of Formula II further comprises a solvent. Solvents include, but are not limited to, water, ethanol, propylene glycol, glycerin, MCT (medium chain triglycerides), vegetable oil, legume oil, or nut oil. Exemplarily oils include, but are not limited to, sunflower oil, olive oil, canola oil, vegetable oil, sesame oil, and avocado oil.

In another embodiment, the solvent comprises ethanol.

In another embodiment, the solvent comprises a seed oil, e.g., sunflower oil.

In another embodiment, the solvent is present at a concentration of from about 90% w/w to about 95% w/w, from about 90% w/w to about 97% w/w, from about 90% w/w to about 98% w/w, from about 90% w/w to about 99% w/w, from about 90% w/w to about 99.99% w/w, from about 95% w/w to about 97% w/w, from about 95% w/w to about 98% w/w, from about 95% w/w to about 99% w/w, from about 95% w/w to about 99.99% w/w, from about 97% w/w to about 98% w/w, from about 97% w/w to about 99% w/w, from about 97% w/w to about 99.99% w/w, from about 98% w/w to about 99% w/w, from about 98% w/w to about 99.99% w/w, or from about 99% w/w to about 99.99% w/w.

In another embodiment, the solvent is present at a concentration of about 99.77% w/w.

In another embodiment, the composition comprises a compound of Formula I present at a concentration of from about 0.1 ppm to about 10 ppm, e.g., about 0.1 ppm, about 0.5 ppm, about 1 ppm, about 1.5 ppm, about 2 ppm, about 2.5 ppm, about 3 ppm, about 3.5 ppm, about 4 ppm, about 4.5 ppm, about 5 ppm, about 5.5 ppm, about 6 ppm, about 6.5 ppm, about 7 ppm, about 7.5 ppm, about 8 ppm, about 8.5 ppm, about 9 ppm, about 9.5 ppm, or about 10 ppm.

In another embodiment, the composition comprises a compound of Formula I present at a concentration of about 3.5 ppm.

In another embodiment, the composition comprises a compound of Formula I present at a concentration of about 2.5 ppm.

In another embodiment, the composition comprises a compound of Formula II present at a concentration of from about 0.1 ppm to about 10 ppm, e.g., about 0.1 ppm, about 0.5 ppm, about 1 ppm, about 1.5 ppm, about 2 ppm, about 2.5 ppm, about 3 ppm, about 3.5 ppm, about 4 ppm, about 4.5 ppm, about 5 ppm, about 5.5 ppm, about 6 ppm, about 6.5 ppm, about 7 ppm, about 7.5 ppm, about 8 ppm, about 8.5 ppm, about 9 ppm, about 9.5 ppm, or about 10 ppm.

In another embodiment, the composition comprises a compound of Formula II present at a concentration of about 1.25 ppm.

In another embodiment, the composition comprises a compound of Formula II present at a concentration of about 2 ppm.

In another embodiment, the composition comprises a compound of Formula I present at a concentration of from about 0.01% w/w to about 1% w/w, e.g., about 0.01% w/w, about 0.05% w/w, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, or about 1% w/w.

In another embodiment, the composition comprises a compound of Formula I present at a concentration of about 0.2% w/w.

In another embodiment, the composition comprises a compound of Formula I present at a concentration of 0.175% w/w.

In another embodiment, the composition comprises a compound of Formula II present at a concentration of from about 0.005% w/w to about 0.1% w/w, e.g., about 0.005% w/w, about 0.01% w/w, about 0.02% w/w, about 0.03% w/w, about 0.04% w/w, about 0.05% w/w, about 0.06% w/w, about 0.07% w/w, about 0.08% w/w, about 0.09% w/w, or about 0.1% w/w.

In another embodiment, the composition comprises a compound of Formula II present at a concentration of about 0.03% w/w.

In another embodiment, the composition comprises a compound of Formula II present at a concentration of 0.05% w/w.

In another embodiment, the composition comprising a compound of Formula I and a compound of Formula II further comprises one or more compounds of Formula III:

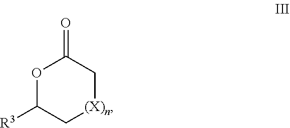

or geometric isomers and/or stereoisomers thereof, wherein:

each X is independently —CH₂—, —CH═CH—CH₂, or —C≡C—CH₂—;

R³ is C₁-C₆ alkyl; and

n is an integer between 6-30, inclusive.

In another embodiment, the compound of Formula III is racemic, i.e., optically inactive.

In another embodiment, the asymmetric carbon atom the compound of Formula III is in the S configuration.

In another embodiment, asymmetric carbon atom of the compound of Formula III is in the R configuration.

In another embodiment, R₃ is methyl, ethyl, or propyl.

In another embodiment, each X is independently —CH₂— or —CH═CH—CH₂—.

In another embodiment, each X is —CH₂—.

In another embodiment, one X group is —CH═CH—CH₂— and the remaining X groups are —CH₂—.

In another embodiment, two X groups are —CH═CH—CH₂— and the remaining X groups are —CH₂—.

In another embodiment, three X groups are —CH═CH—CH₂— and the remaining X groups are —CH₂—.

In another embodiment, four X groups are —CH═CH—CH₂— and the remaining X groups are —CH₂—.

In another embodiment, one X group is —CH═CH—CH₂—, wherein the double bond is an E double bond.

In another embodiment, one X group is —CH═CH—CH₂—, wherein the double bond is an Z double bond.

In another embodiment, two X groups are —CH═CH—CH₂—, wherein one double bond is an E double bond and the other double bond is a Z double bond.

In another embodiment, two X groups are —CH═CH—CH₂—, wherein each double bond is an E double bond.

In another embodiment, two X groups are —CH═CH—CH₂—, wherein each double bond is a Z double bond.

In another embodiment, three X groups are —CH═CH—CH₂—, wherein one double bond is an E double bond and the other double bonds are Z double bonds.

In another embodiment, three X groups are —CH═CH—CH₂—, wherein one double bond is an Z double bond and the other double bonds are E double bonds.

In another embodiment, three X groups are —CH═CH—CH₂—, wherein each double bond is an E double bond.

In another embodiment, three X groups are —CH═CH—CH₂—, wherein each double bond is a Z double bond.

In another embodiment, four X groups are —CH═CH—CH₂—, wherein one double bond is an E double bond and the other double bonds are Z double bonds.

In another embodiment, four X groups are —CH═CH—CH₂—, wherein two double bonds are E double bonds and the other double bonds are Z double bonds.

In another embodiment, four X groups are —CH═CH—CH₂—, wherein three double bonds are E double bonds and the other double bond is a Z double bond.

In another embodiment, four X groups are —CH═CH—CH₂—, wherein each double bond is an E double bond.

In another embodiment, four X groups are —CH═CH—CH₂—, wherein each double bond is a Z double bond.

In another embodiment, n is 6. In another embodiment, n is 7. In another embodiment, n is 8. In another embodiment, n is 9. In another embodiment, n is 10. In another embodiment, n is 11. In another embodiment, n is 12. In another embodiment, n is 13. In another embodiment, n is 14. In another embodiment, n is 15. In another embodiment, n is 16. In another embodiment, n is 17. In another embodiment, n is 18. In another embodiment, n is 19. In another embodiment, n is 20. In another embodiment, n is 21. In another embodiment, n is 22. In another embodiment, n is 23. In another embodiment, n is 24. In another embodiment, n is 25. In another embodiment, n is 26. In another embodiment, n is 27. In another embodiment, n is 28. In another embodiment, n is 29. In another embodiment, n is 30.

In another embodiment, the composition comprises a mixture of two or more compounds of Formula III, wherein the compounds have different n values and different R³ moieties. In another embodiment, the compounds have different n values, different R³ moieties, and the same R/S configuration at the asymmetric carbon atom. In another embodiment, the compounds have the same n values, the same R³ moieties, and different R/S configurations at the asymmetric carbon atom. In another embodiment, the compounds have different n values, different R³ moieties, and different R/S configurations at the asymmetric carbon atom. In another embodiment, the mixture is a racemic mixture of compounds.

In another embodiment, the composition comprises one or more compounds of Formula III selected from:

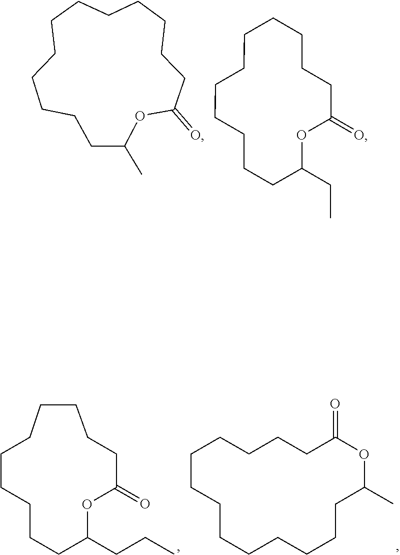

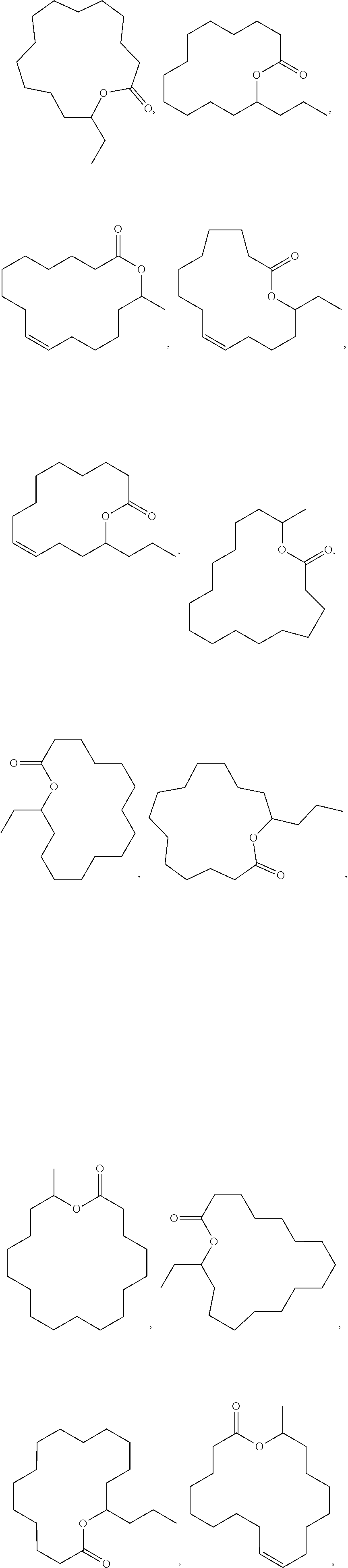

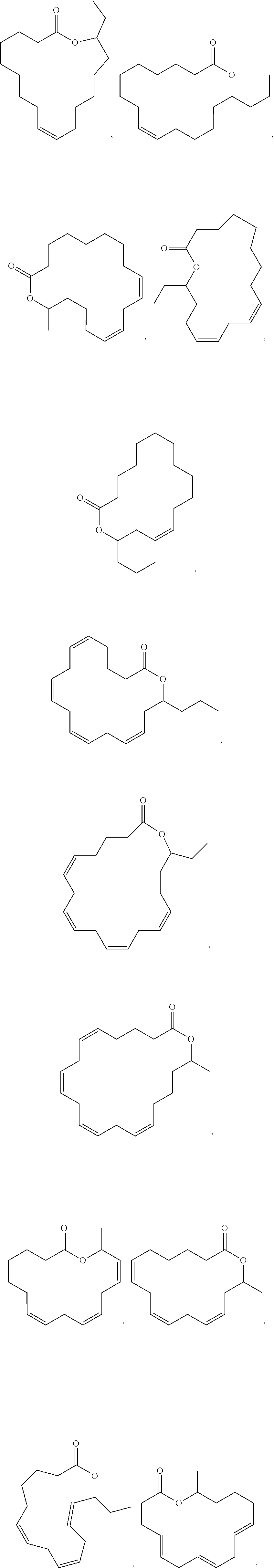

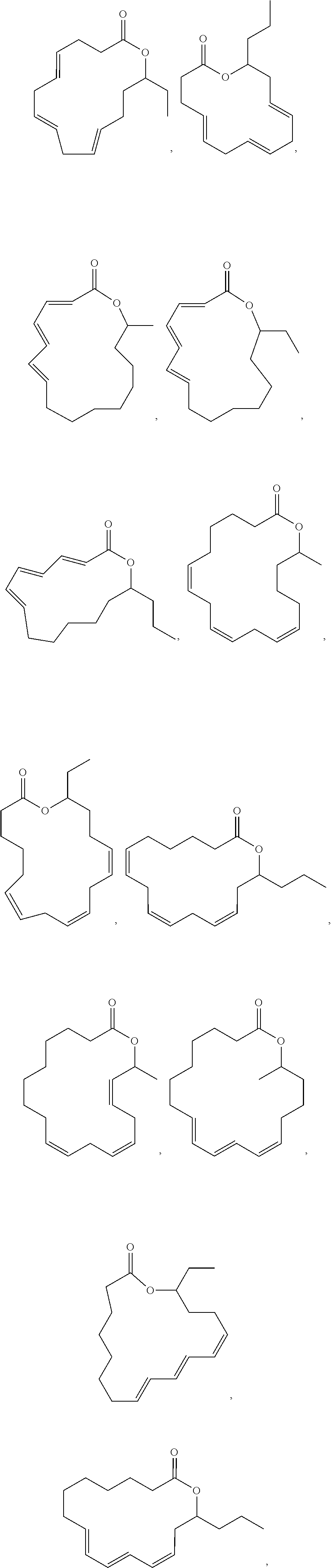

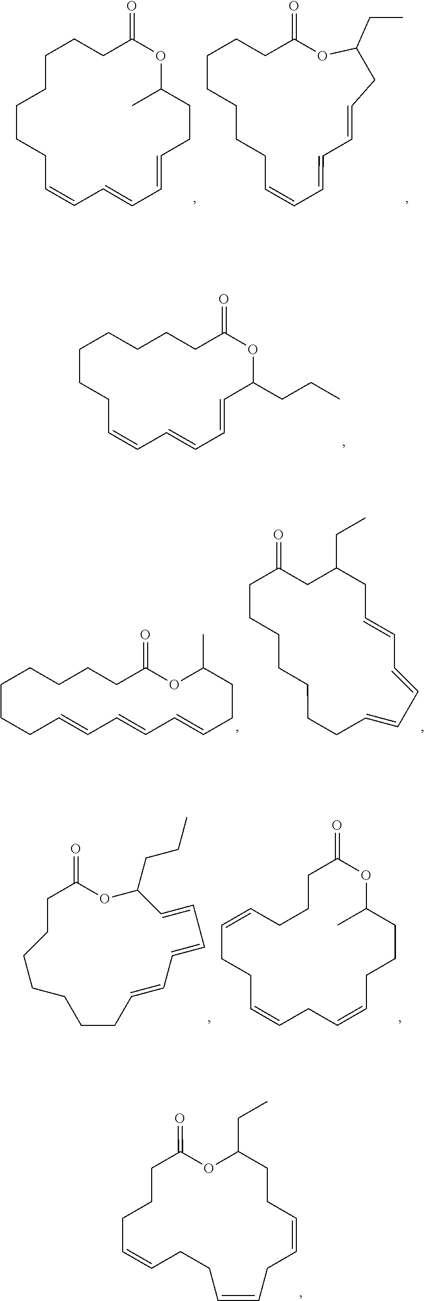

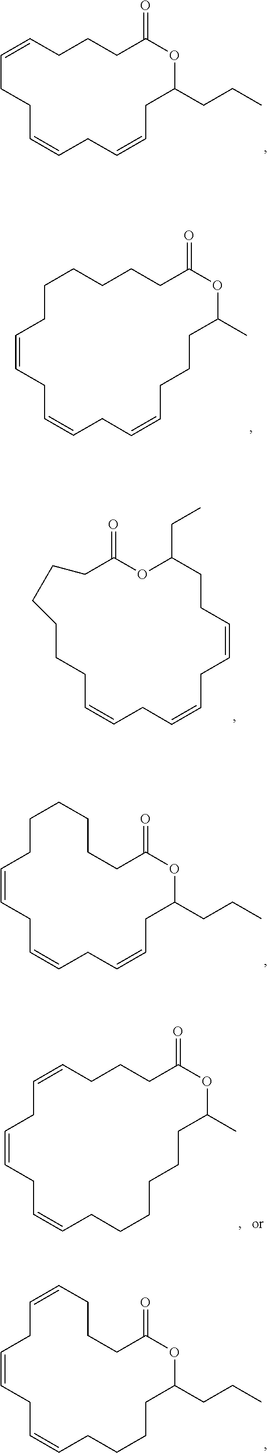 [SMILES: CCCC1C/C=C\C/C=C\CC/C=C\CCCC(=O)O1 ; CC1CCC/C=C\C/C=C\C/C=C\CCCCCCC(=O)O1 ; CCC1CC/C=C\C/C=C\C/C=C\CCCCCCC(=O)O1 ; CCCC1C/C=C\C/C=C\C/C=C\CCCCCCC(=O)O1 ; CC1CCCCCC/C=C\C/C=C\C/C=C\CCCC(=O)O1 ; CCCC1CCCC/C=C\C/C=C\C/C=C\CCCC(=O)O1]

or a combination thereof, or geometric isomers and/or stereoisomers thereof.

In another embodiment, the composition comprises one or more compounds of Formula III selected from:

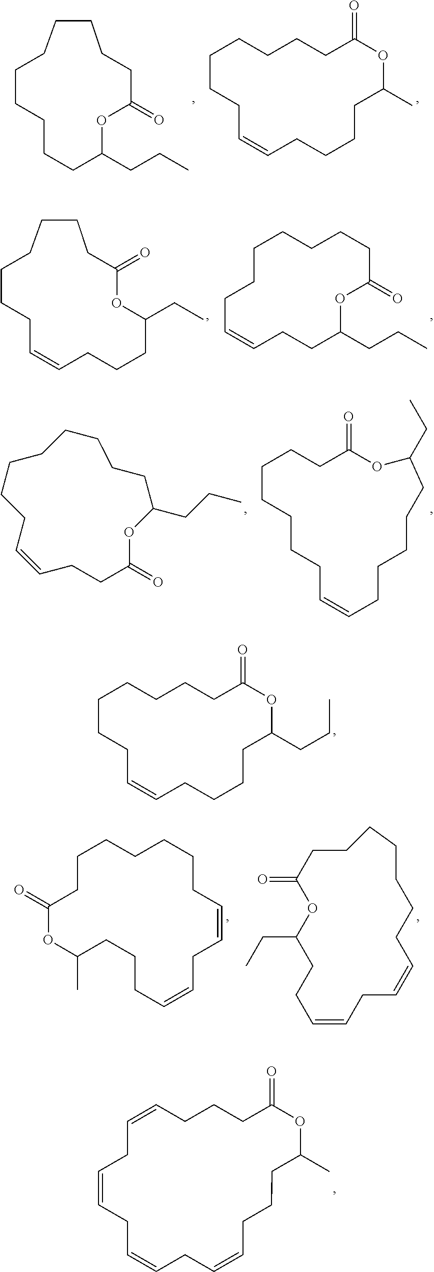

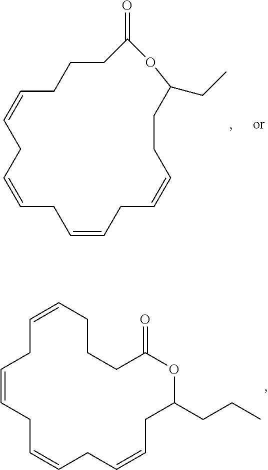 [SMILES: CCC1CC/C=C\C/C=C\C/C=C\C/C=C\CCCC(=O)O1 ; CCCC1C/C=C\C/C=C\C/C=C\C/C=C\CCCC(=O)O1]

or a combination thereof, or geometric isomers and/or stereoisomers thereof.

In another embodiment, the composition comprises one or more compounds of Formula III selected from:

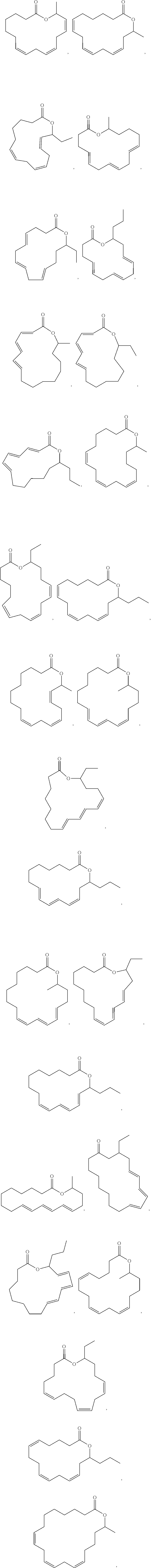

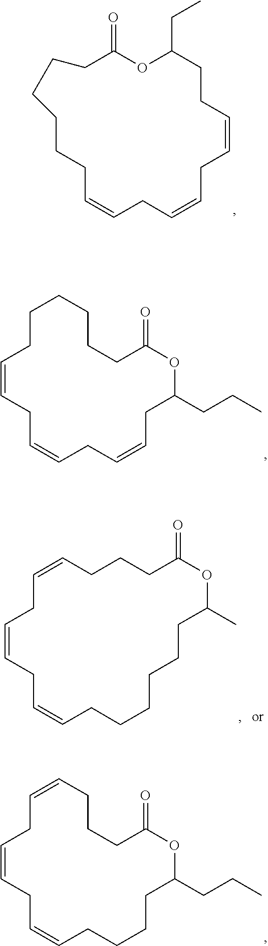 [SMILES: CCC1CC/C=C\C/C=C\C/C=C\CCCCCCC(=O)O1 ; CCCC1C/C=C\C/C=C\C/C=C\CCCCCCC(=O)O1 ; CC1CCCCCC/C=C\C/C=C\C/C=C\CCCC(=O)O1 ; CCCC1CCCC/C=C\C/C=C\C/C=C\CCCC(=O)O1]

or a combination thereof, or geometric isomers and/or stereoisomers thereof.

In another embodiment, the composition comprises one compound of Formula III.

In another embodiment, the composition comprises two compounds of Formula III

In another embodiment, the composition comprises three compounds of Formula III

The term “C₁₋₆ alkyl” as used herein refers to a straight- or branched-chain aliphatic hydrocarbon containing one to six carbon atoms. Non-limiting exemplary C₁₋₆ alkyl groups include methyl, ethyl, propyl, iso-propyl, butyl, sec-butyl, tert-butyl, iso-butyl, 3-pentyl, and hexyl. In one embodiment, the C₁₋₆ alkyl is methyl. In another embodiment, the C₁₋₆ alkyl is ethyl. In another embodiment, the C₁₋₆ alkyl is propyl. In another embodiment, the C₁₋₆ alkyl is iso-propyl. In another embodiment, the C₁₋₆ alkyl is tert-butyl.

The term “C₈₋₁₆ alkyl” as used herein refers to a straight- or branched-chain aliphatic hydrocarbon containing eight to sixteen carbon atoms. In one embodiment, the C₈₋₁₆ alkyl is a straight-chain aliphatic hydrocarbon containing eight to sixteen carbon atoms. In one embodiment, the C₈₋₁₆ alkyl is a branched-chain aliphatic hydrocarbon containing eight to sixteen carbon atoms. Non-limiting exemplary C₈₋₁₆ alkyl groups include octyl, nonyl, decyl, undecyl, dodecyl, tridecyl, tetradecyl, pentadecyl, and hexadecyl. In one embodiment, the C₈₋₁₆ alkyl is nonyl. In another embodiment, the C₈₋₁₆ alkyl is undecyl.

Compounds having Formulae I-III contain an asymmetric carbon atom and may thus give rise to enantiomers, diastereomers, and other stereoisomeric forms. Unless specified otherwise, the present disclosure encompasses the use of all such possible forms, as well as their racemic and resolved forms and mixtures thereof. The individual enantiomers can be separated according to methods known in the art in view of the present disclosure. Compounds of Formula III also contain multiple olefinic double bonds or other centers of geometric asymmetry and, unless specified otherwise, the present disclosure encompasses the use of all such possible geometric forms and mixtures thereof. For example, it is intended that a compound described herein includes both E and Z geometric isomers. All tautomers are also encompassed by the present disclosure.

As used herein, the term “stereoisomers” is a general term for all isomers of an individual molecule that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography, or optical polarimetry.

The use of the terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

### Methods of Use

In another embodiment, the disclosure provides methods of enhancing the flavor, texture, or richness of a plant-based meat product or plant-based dairy product comprising admixing a composition comprising:

(a) a compound of Formula I:

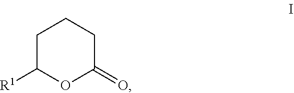

or a stereoisomer thereof, wherein R¹ is C₈-C₁₆ alkyl; and

(b) a compound of Formula II:

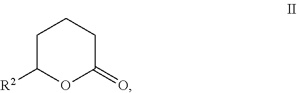

or a stereoisomer thereof, wherein R² is C₈-C₁₆ alkyl,

with the proviso that R¹ does not equal R²;

with the plant-based meat or plant-based dairy product.

In another aspect, the composition further comprises one or more compounds of Formula III:

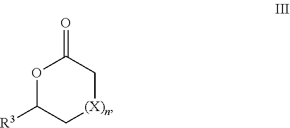

or geometric isomers and/or stereoisomers thereof, wherein:

each X is independently —CH₂—, —CH═CH—CH₂, or —C≡C—CH₂—;

R³ is C₁-C₆ alkyl; and

n is an integer between 6-30, inclusive.

In another embodiment, the concentration of the composition within the plant-based meat product or plant-based dairy product is from about 0.08% to about 0.5%, e.g., about 0.08%, about 0.09%, about 0.1%, about 0.2%, about 0.3%, about 0.4%, or about 0.5%.

The disclosure also provides the following particular embodiments relating to methods and uses comprising a composition comprising a compound of Formula I, a compound of Formula II, and optionally one or more compounds of Formula III.

Embodiment I. A method of enhancing the flavor, texture, or richness of a plant-based meat product or plant-based dairy product comprising admixing a composition comprising:

(a) a compound of Formula I:

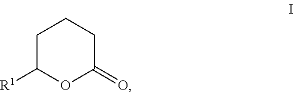

or a stereoisomer thereof, wherein R¹ is C₈-C₁₆ alkyl; and

(b) a compound of Formula II:

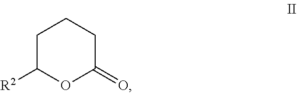

or a stereoisomer thereof, wherein R¹ is C₈-C₁₆ alkyl,

with the proviso that R¹ does not equal R²;

with the plant-based meat or plant-based dairy product.

Embodiment II. The method of Embodiment I, wherein the composition further comprises one or more compounds of Formula III:

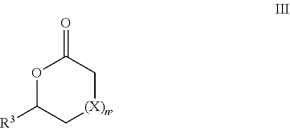

or geometric isomers and/or stereoisomers thereof, wherein:

each X is independently —CH₂—, —CH═CH—CH₂, or —C≡C—CH₂—;

R³ is C₁-C₆ alkyl; and

n is an integer between 6-30, inclusive.

Embodiment III. The method of Embodiment I or II, wherein the concentration of the composition within the plant-based meat product or plant-based dairy product is from about 0.08% to about 0.5%.

Embodiment IV. The method of Embodiment III, wherein the concentration of the composition within the plant-based meat product or plant-based dairy product is about 0.2%.

Embodiment V. The method of any one of Embodiments I-IV for enhancing the flavor, texture, or richness of a plant-based meat product.

Embodiment VI. The method of Embodiment V, wherein the plant-based meat product is formulated to mimic beef, pork, chicken, or fish.

Embodiment VII. The method of Embodiment VI, wherein the plant-based meat product is formulated to mimic a beef burger patty.

Embodiment VIII. The method of any one of Embodiments I-IV for enhancing the flavor, texture, or richness of a plant-based dairy product.

Embodiment IX. The method of Embodiment VIII, wherein the plant-based dairy product is formulated to mimic milk, cheese, ice cream, or yogurt.

Embodiment X. The method of Embodiment VIII, wherein the plant-based dairy product is almond milk, barley milk, oat milk, coconut milk, soy milk, legume milk, nut milk, or grain milk.

Embodiment XI. The method of Embodiment X, wherein the plant-based dairy product is almond milk, barley milk, oat milk, coconut milk, or soy milk.

Embodiment XII. The method of Embodiment XI, wherein the plant-based dairy product is almond milk.

Embodiment XIII. A composition comprising:

(a) a compound of Formula I:

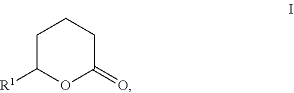

or a stereoisomer thereof, wherein R¹ is C₈-C₁₆ alkyl; and

(b) a compound of Formula II:

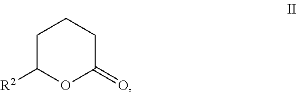

or a stereoisomer thereof, wherein R¹ is C₈-C₁₆ alkyl,

with the proviso that R¹ does not equal R²;

for use in enhancing the flavor, texture, or richness of a plant-based meat product or plant-based dairy product.

Embodiment XIV. The composition of Embodiment XIII, wherein the composition further comprises one or more compounds of Formula III:

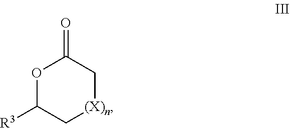

or geometric isomers and/or stereoisomers thereof, wherein:

each X is independently —CH₂—, —CH═CH—CH₂, or —C≡C—CH₂—;

R³ is C₁-C₆ alkyl; and

n is an integer between 6-30, inclusive.

Embodiment XV. The method of Embodiment XIII or XIV, wherein the concentration of the composition within the plant-based meat product or plant-based dairy product is from about 0.08% to about 0.5%.

Embodiment XVI. The method of Embodiment XV, wherein the concentration of the composition within the plant-based meat product or plant-based dairy product is about 0.2%.

Embodiment XVII. The method of any one of Embodiments XIII-XVI for enhancing the flavor, texture, or richness of a plant-based meat product.

Embodiment XVIII. The method of Embodiment XVII, wherein the plant-based meat product is formulated to mimic beef, pork, chicken, or fish.

Embodiment XIX. The method of Embodiment XVIII, wherein the plant-based meat product is formulated to mimic a beef burger patty.

Embodiment XX. The method of any one of Embodiments XIII-XVI for enhancing the flavor, texture, or richness of a plant-based dairy product.

Embodiment XXI. The method of Embodiment XX, wherein the plant-based dairy product is formulated to mimic milk, cheese, ice cream, or yogurt.

Embodiment XXII. The method of Embodiment XX, wherein the plant-based dairy product is a legume milk, nut milk, or grain milk.

Embodiment XXIII. The method of Embodiment XXII, wherein the plant-based dairy product is almond milk, barley milk, oat milk, coconut milk, or soy milk.

Embodiment XXIV. The method of Embodiment XIII, wherein the plant-based dairy product is almond milk.

### EXAMPLES

### Example 1

### Flavor Solution for Plant-Based Burger Patty

Compound I-1 and Compound II-1 were dissolved in sunflower oil using heat and a stir plate. The final concentrations in the solution are shown in Table 2.

TABLE 2

| | Concentration of | Concentration of | Concentration of |
|---|---|---|---|
| Solution | Compound I-1 | Compound II-1 | sunflower oil |
| Combination A | 0.2% w/w | 0.03% w/w | 99.77% w/w |
| Combination B | 0.175% w/w | 0.05% w/w | 99.77% w/w |

0.2% w/w of Combination A or Combination B was admixed with a plant-based burger patty to give the desired effect of a richness enhancer.

### Example 2

### Flavor Solution for Almond Milk

Compound I-1 and Compound II-1 were dissolved in ethanol and admixed with almond milk at the ratios and concentrations shown in Table 3.

| | | | Ratio of |
|---|---|---|---|
| Almond Milk | Concentration of | Concentration of | Compound I-1 |
| Composition | Compound I-1 | Compound II-1 | to Compound II-1 |
| Composition A | 3.5 ppm | 1.25 ppm | 2.8 |
| Composition B | 2.5 ppm | 2.0 ppm | 1.25 |

1. A composition comprising:
(a) a compound of Formula I: 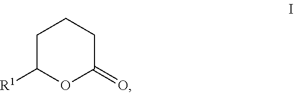
or a stereoisomer thereof, wherein R¹ is C₈-C₁₆ alkyl; and
(b) a compound of Formula II: 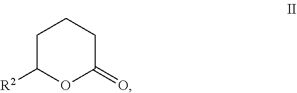
or a stereoisomer thereof, wherein R² is C₈-C₁₆ alkyl,
with the proviso that R¹ does not equal R².

2.-3. (canceled)

4. The composition of claim 1, wherein the compound of Formula I is:

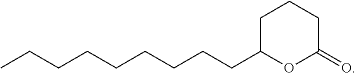 [SMILES: CCCCCCCCCC1CCCC(=O)O1]

5. (canceled)

6. The composition of claim 1, wherein the compound of Formula II is:

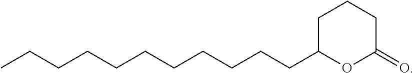 [SMILES: CCCCCCCCCCCC1CCCC(=O)O1]

7. The composition of claim 1, wherein the ratio of the compound of Formula I to the compound of Formula II is from about 1:1 to about 10:1.

8.-9. (canceled)

10. The composition of claim 1, wherein the composition further comprises a solvent.

11. The composition of claim 10, wherein the solvent is water, ethanol, propylene glycol, glycerin, medium chain triglycerides, vegetable oil, legume oil, nut oil, or seed oil.

12. The composition of claim 1, wherein the concentration of the compound of Formula I is from about 0.1 ppm to about 10 ppm.

13.-15. (canceled)

16. The composition of claim 1, wherein the concentration of the compound of Formula II is from about 0.1 to about 10 ppm.

17.-19. (canceled)

20. The composition of claim 1, wherein the composition is admixed with a plant-based dairy product.

21.-25. (canceled)

26. The composition of claim 1, wherein the ratio of the compound of Formula I to the compound of Formula II is from about 1:3 to about 12:1.

27.-28. (canceled)

29. The composition of claim 1, wherein the concentration of the compound of Formula I is from about 0.01% w/w to about 1% w/w.

30.-32. (canceled)

33. The composition of claim 1, wherein the concentration of the compound of Formula II is from about 0.005% w/w to about 0.1% w/w.

34.-36. (canceled)

37. The composition of claim 1, further comprising one or more compounds of Formula III: 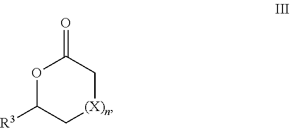
or geometric isomers and/or stereoisomers thereof, wherein:
each X is independently —CH₂—, —CH═CH—CH₂, or —C≡C—CH₂—;
R³ is C₁-C₆ alkyl; and
n is an integer between 6-30, inclusive.

38.-47. (canceled)

48. The composition of claim 37, wherein the one or more compounds of Formula III are: 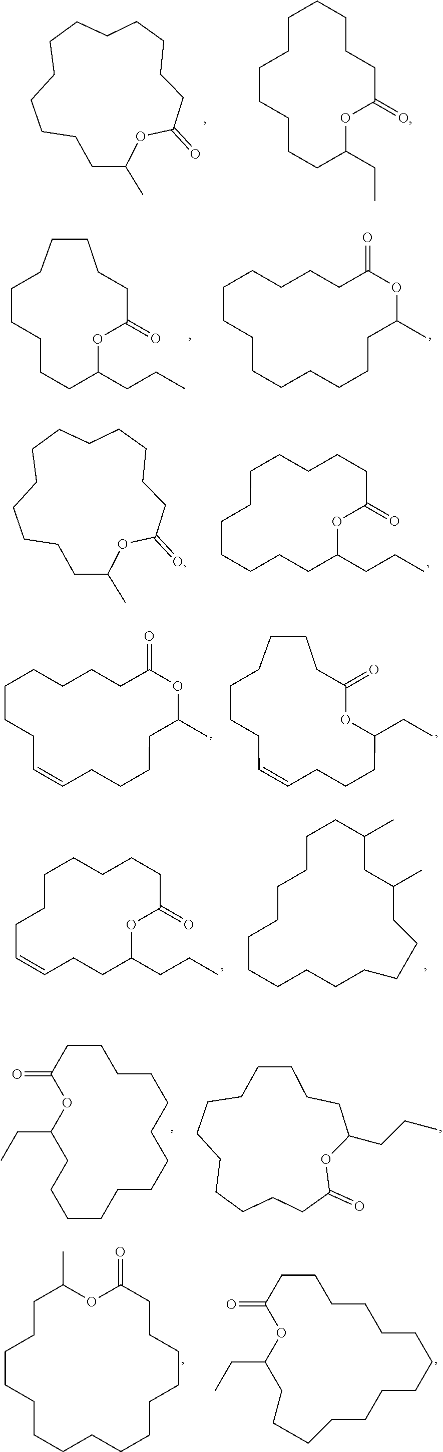 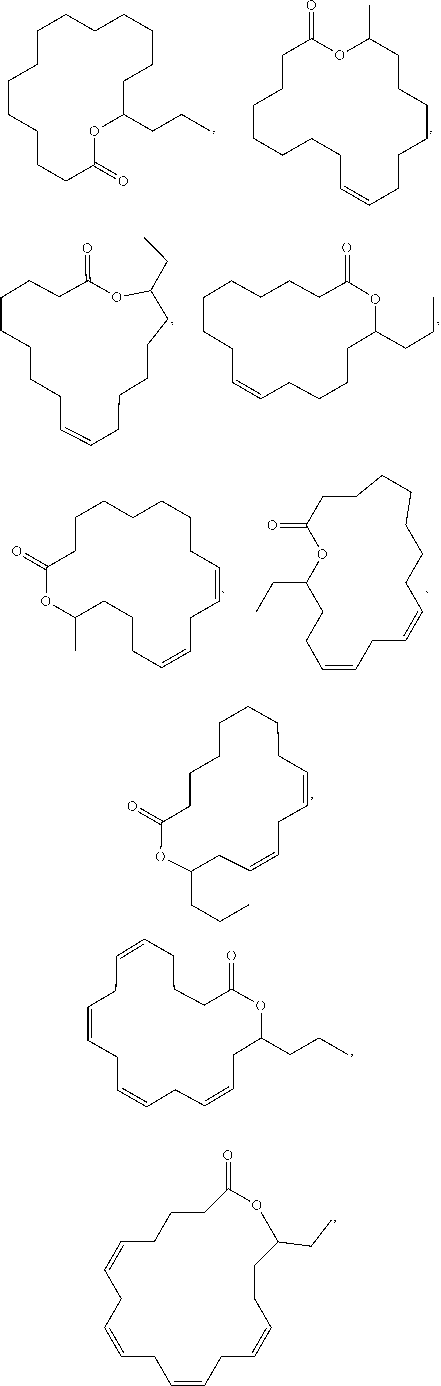 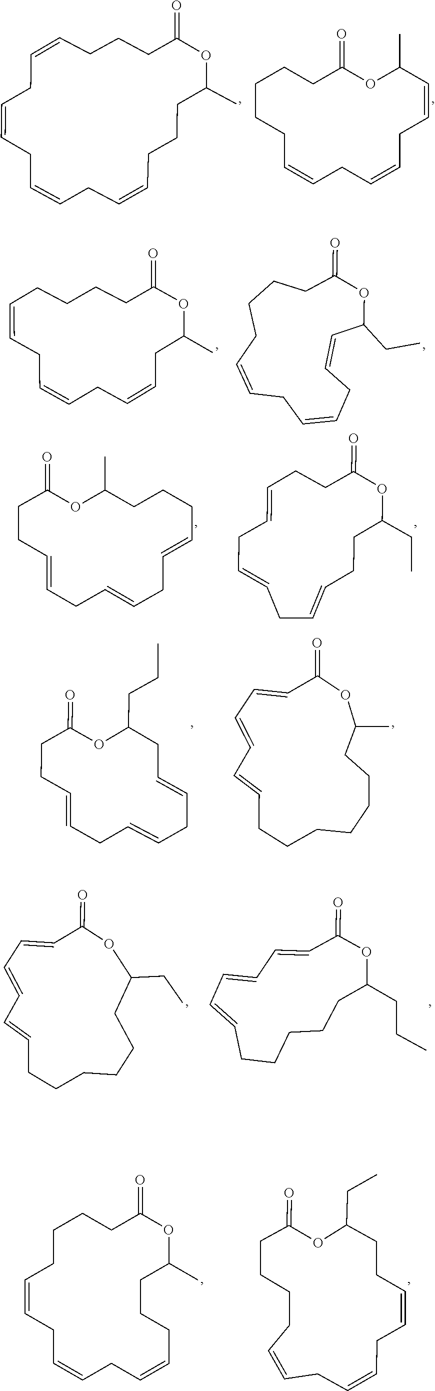 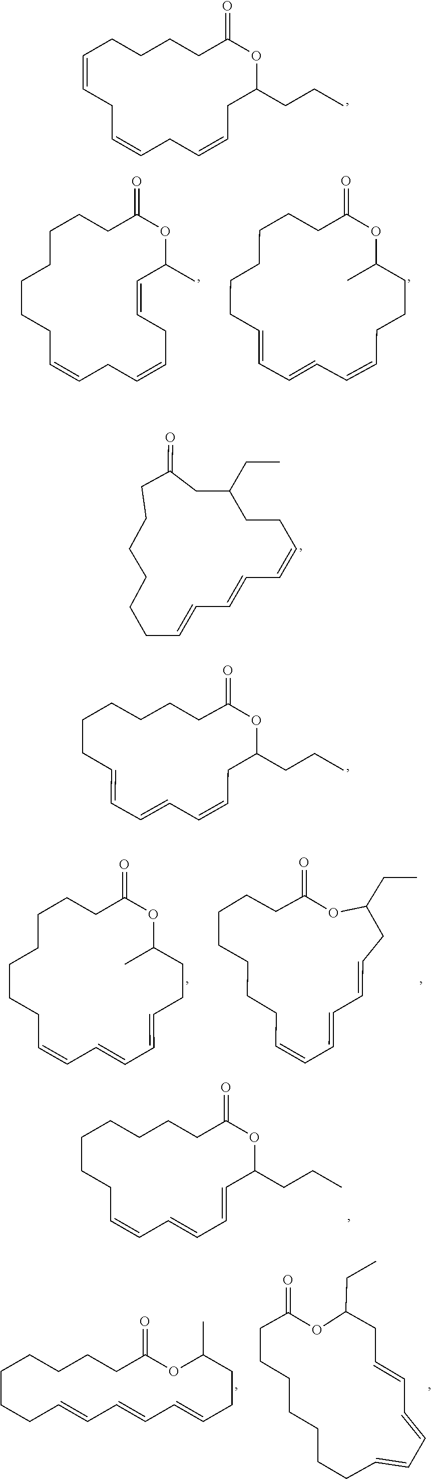 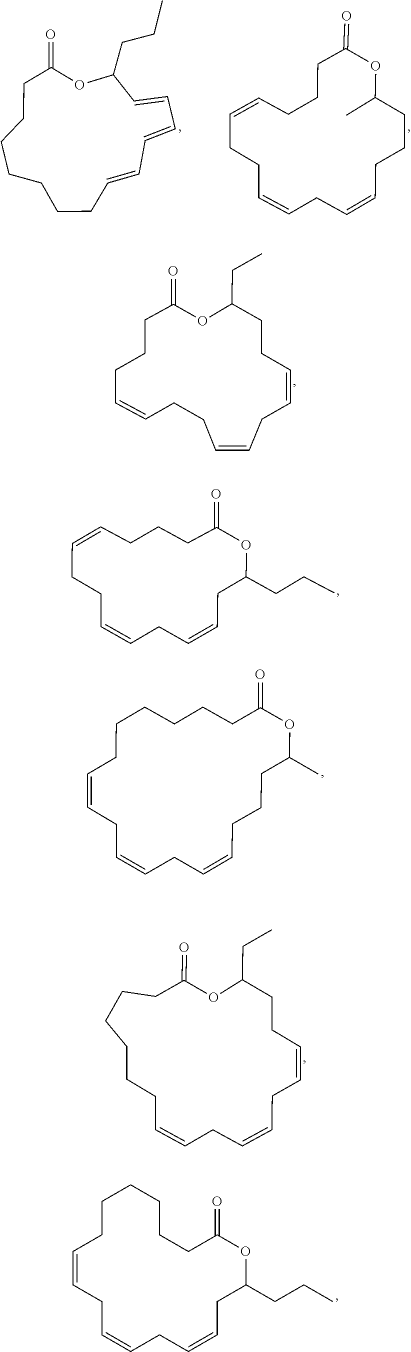 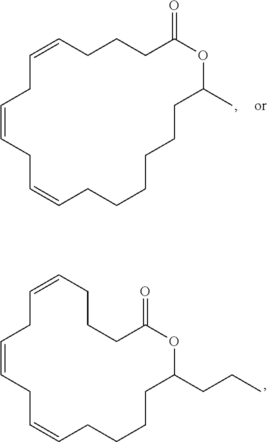 [SMILES: CC1CCCCCC/C=C\C/C=C\C/C=C\CCCC(=O)O1 ; CCCC1CCCC/C=C\C/C=C\C/C=C\CCCC(=O)O1]
or a combination thereof,
or geometric isomers and/or stereoisomers thereof.

49.-50. (canceled)

51. A method of enhancing the flavor, texture, or richness of a plant-based meat product or plant-based dairy product comprising admixing the composition of claim 1 with the plant-based meat or plant-based dairy product.

52. The method of claim 51, wherein the concentration of the composition within the plant-based meat product or plant-based dairy product is from about 0.08% to about 0.5%.

53. (canceled)

54. The method of claim 51 for enhancing the flavor, texture, or richness of a plant-based meat product.

55. The method of claim 54, wherein the plant-based meat product is formulated to mimic beef, pork, chicken, or fish.

56. (canceled)

57. The method of claim 51 for enhancing the flavor, texture, or richness of a plant-based dairy product.

58. The method of claim 57, wherein the plant-based dairy product is formulated to mimic milk, cheese, ice cream, or yogurt.

59.-61. (canceled)
